(19) United States
(12) Patent Application Publication
(10) Pub. No.: US 2022/0403432 A1
(43) Pub. Date: Dec. 22, 2022

(54) METHOD FOR PREPARING CHONDROITIN SULFATE OLIGOSACCHARIDES WITH C5 PROTEIN TARGETING PROPERTY, AND APPLICATION THEREOF

(71) Applicant: SHANDONG UNIVERSITY, Jinan, Shandong (CN)

(72) Inventors: Lian LI, Jinan (CN); Chen YU, Jinan (CN); Hengchang ZANG, Jinan (CN); Shuang QUAN, Jinan (CN); Yan LI, Jinan (CN); Fengshan WANG, Jinan (CN)

(73) Assignee: SHANDONG UNIVERSITY, Jinan, Shandong (CN)

(21) Appl. No.: 17/772,359

(22) PCT Filed: Feb. 3, 2021
(86) PCT No.: PCT/CN2021/075082

§ 371 Date: Apr. 27, 2022

(30) Foreign Application Priority Data
May 26, 2020 (CN) 202010454657.0

(51) Int. Cl. C12P 19/26; G01N 30/02

(52) U.S. Cl. CPC C12P 19/26; G01N 30/02

(57) ABSTRACT

A method for preparing chondroitin sulfate oligosaccharides with a C5 protein targeting property, and an application thereof, relating to the technical field of biomedicine. The method uses squid chondroitin sulfate as a raw material to prepare chondroitin sulfate oligosaccharides using enzymatic hydrolysis, using the method to screen the activity of various types of oligosaccharides in order to obtain the fragment with the best activity; the method is highly scientific and feasible. The method further uses the screened chondroitin sulfate disaccharides; the mechanism of chondroitin sulfate disaccharides in the treatment of osteoarthritis by regulating the complement system is explored and revealed by in vitro and in vivo experiments to finally provide a new way of treating osteoarthritis, thus having good practical application value.

### CROSS-REFERENCES TO RELATED APPLICATIONS

This application claims priority benefits to Chinese Patent Application No. 202010454657.0, filed 26 May 2020, the contents of which are incorporated herein by reference.

### TECHNICAL FIELD

The invention belongs to the technical field of biomedicine, and in particular relates to a method for preparing chondroitin sulfate oligosaccharides with C5 protein targeting property, and application thereof.

### BACKGROUND

The information disclosed in the background is intended to increase the understanding of the overall background of the application, and the disclosure should not necessarily be regarded as an acknowledgement or in any form implying that the information has become prior art known to those of ordinary skill in the art.

Osteoarthritis (OA) is a degenerative joint disease caused by the mechanical and biological degradation of articular cartilage, its prevalence is increasing especially in the elderly, and the incidence is growing younger, seriously affecting the health and quality of life of patients. Therefore, the prevention and treatment of OA is particularly important. The pathogenesis of OA is complex. It is generally believed that OA is due to a combination of genetic, metabolic, biochemical, biomechanical and other factors, as well as mild and moderate inflammation resulting in cartilage damage. However, to date, there is no complete cure for OA. Non-steroidal anti-inflammatory drugs (NSAIDs) are clinically used to relieve patients' pain, but cannot help cure diseases, and some patients will have strong adverse reactions such as gastrointestinal bleeding and cardiovascular diseases after using NSAIDs. As research progresses, more and more results suggest that the immune system is included in the overall pathogenesis of OA and that the complement system plays a central role in the process. Studies have shown that the initial cartilage damage during the pathogenesis of OA leads to the release of components of the cartilage extracellular matrix, such as cartilage oligomeric matrix proteins, which activate the complement system. The complement system is mainly composed of the classical pathway (CP), the alternative pathway (AP) and the lectin pathway (LP). On the one hand, the three pathways converge when complement proteins C3 and C5 are formed, then C3 and C5 invertase are produced respectively, which induce C3a and C5a production respectively, thus producing pro-inflammatory cytokines; On the other hand, complement activation leads to the formation of membrane attack complex (MAC) on the surface of chondrocytes. MAC can not only insert into cells to form cavities to promote chondrocyte lysis and death, but also promote the secretion of IL-10 and TNF-α, thus activating NF-κB, MAPK, and other signal pathways, releasing MMPs and ADAMTSs, resulting in cartilage injury and forming OA. In addition, the activation of the complement system occurs before the level of inflammatory factors such as IL-10 and TNF-α increases, which further indicates the importance of complement. These findings provide further evidence that activation of the complement system is a central factor in the pathogenesis of OA. In summary, the complement system may be a new drug target for the treatment of OA, and regulation of it may provide a new strategy for OA.

Chondroitin sulfate (CS) represents a class of unbranched linear anionic glycosaminoglycans (GAGs) composed of disaccharide units named D-glucuronic acid and N-acetyl-D-galactosamine. CS has many clinical applications, such as anticoagulant, anti-tumor, anti-inflammatory and anti-complement activities. In Europe, CS is recommended by the European League against Rheumatism (EULAR) as a slow-acting drug for the treatment of OA. Most studies have shown that CS can achieve the treatment of OA by intervening in the inflammatory system, and it is a good multi-targeted natural polysaccharide potential drug. However, the mechanism of CS in OA treatment through the regulation of complement system has not been reported, and the interaction mechanism between CS and a series of complement components is still unclear. This is because the structure of CS is more complex compared to the other GAGs such as heparin, and is influenced by a series of factors such as cartilage origin and age. In addition, the inventors found that CS with complete chain length is difficult to pass through the gastric and intestinal mucosa, while low molecular weight chondroitin sulfate (LMWCS) can pass through the intestinal mucosa. LMWCS has better bioavailability and therapeutic effect on type II collagen-induced rheumatoid arthritis compared to intact CS, but the efficacy of different sizes of LMWCS varies. Therefore, screening CS fragments with specific anti-complement activity and targeting, studying the mechanism between CS fragments and complement system are of great significance for the treatment of OA.

### SUMMARY

The present invention provides method for preparing chondroitin sulfate (CS) oligosaccharides with C5 protein targeting property, and application thereof. The CS oligosaccharides are prepared by enzymatic hydrolysis using squid-derived CS as raw material, the method of the invention is used for screening and obtaining the fragments with C5 protein targeting and best activity, this method has high scientificity and strong feasibility. The present invention further utilizes the screened CS disaccharide to explore and reveal its stronger C5 protein targeting property and the mechanism for treating OA by regulating a complement system through in vitro and in vivo experiments, thereby finally providing a new way for treating OA. In conclusion, the invention has good practical application value.

In order to achieve the above objectives, the present invention adopts the following technical solutions:

according to a first aspect of the present invention, there is provided a method for preparing CS oligosaccharides with C5 protein targeting properties, the method comprising:

carrying out enzymatic hydrolysis of CS and separating enzymatic hydrolysis products to obtain different oligosaccharide components (oligosaccharides);

verifying the oligosaccharides using hemolysis assay and cell proliferation assay for preliminary investigation and screening of CS oligosaccharides.

Specifically, the CS is derived from squid.

The enzymatic hydrolysis is performed using CS ABC enzymes.

The enzymatic hydrolysis product is separated by adopting a column chromatography method, the column used is in column chromatography such as Bio-Gel P10 column, which is more effective for separating the oligosaccharide components.

Preferably, the CS oligosaccharides include, but are not limited to, CS disaccharides, CS tetrasaccharides, CS hexasaccharides, CS octasaccharides and CS decasaccharides; preferably CS disaccharide. The CS disaccharide obtained by the preparation and screening method of the present invention was experimentally verified to have optimal C5 protein targeting.

According to a second aspect of the present invention, there is provided a CS oligosaccharide, the CS oligosaccharide was prepared by the above method.

According to a third aspect of the present invention, there is provided an application of the above-mentioned CS oligosaccharide in the preparation of OA therapeutic medicines.

Beneficial technical effects of one or more of the above technical features or technical solutions:

1. The method for screening CS oligosaccharide with C5 protein targeting provided by the present invention has a clear principle, easy experimental operation, and high confidence of the results.

2. The CS disaccharide provided in the present invention has low molecular weight, good penetration, high oral bioavailability, good C5 protein targeting, can promote the proliferation of mouse chondrocytes, significantly inhibit the formation of C3a, C4b, C5 complement and MAC in the complement pathway and the expression of MAC-induced related proteins, and therefore has good value for practical application.

### BRIEF DESCRIPTION OF THE DRAWINGS

The accompanying drawings, which are incorporated in and constitute a part of this application, are included to provide a further understanding of the application, and the description of the exemplary embodiments and illustrations of the application are intended to explain the application and are not intended to limit the application.

FIG. 1 shows the chromatographic separation results and mass spectra of each oligosaccharide component of chondroitin sulfate in Example 1, wherein: a is the chromatographic separation result of CS, b is the mass spectra of component A, c is the mass spectra of component B, d is the mass spectra of component C, e is the mass spectra of component D, and f is the mass spectra of component E.

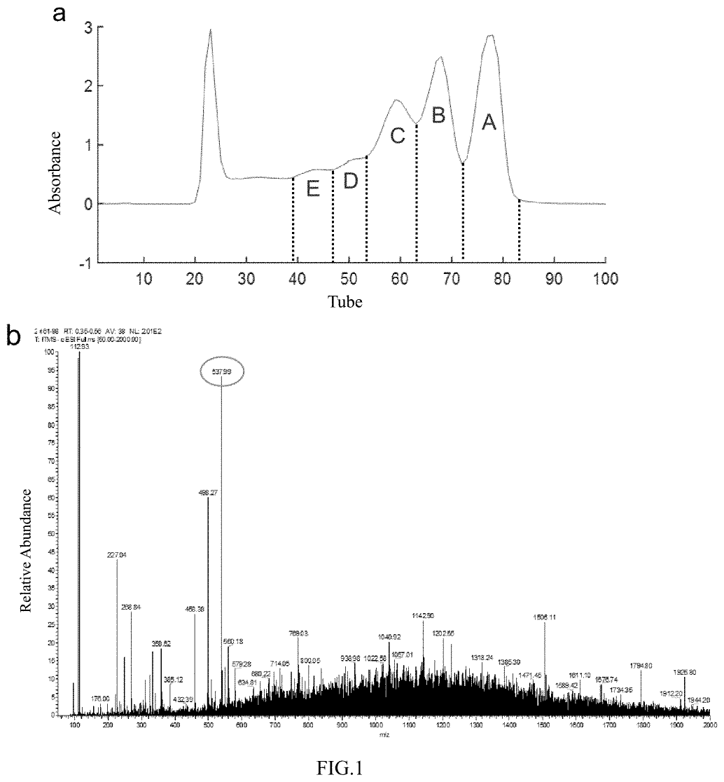

FIG. 2 shows the anti-complement activity of CS disaccharide, tetrasaccharide, hexasaccharide and octasaccharide at different concentrations in Example 2.

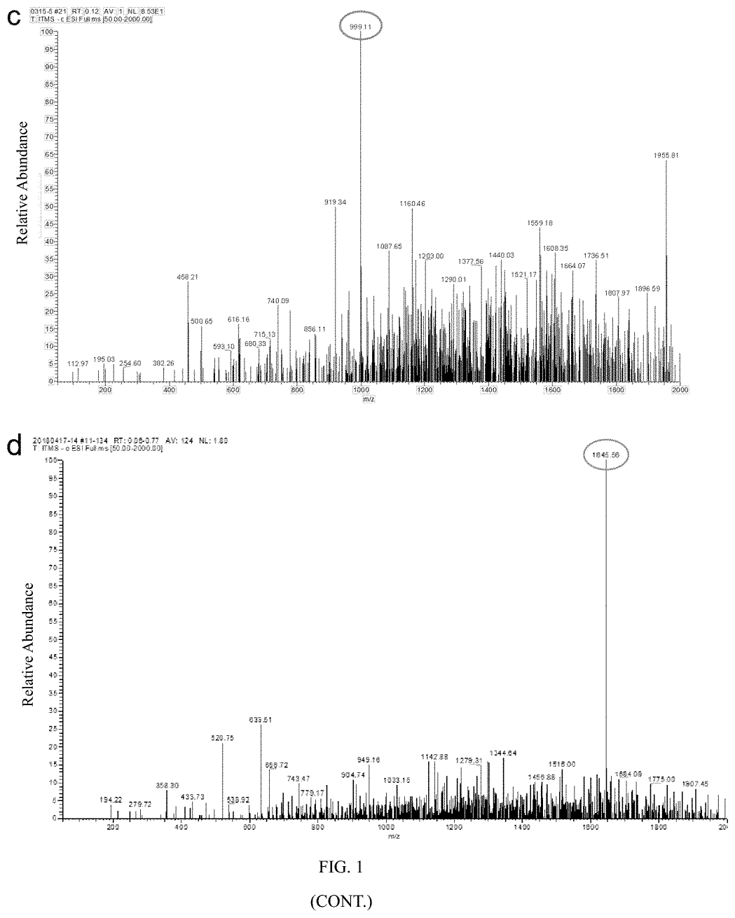

FIG. 3 shows the chondrocyte proliferative activity promoted by different concentrations of CS oligosaccharide in Example 3; a shows the relative cell numbers of mouse chondrocytes after 24 h of culture under different concentrations of CS disaccharide conditions. * P<0.05, ns: no significant difference; b is the pro-proliferation rate curve of mouse chondrocytes by different concentrations of CS disaccharide.

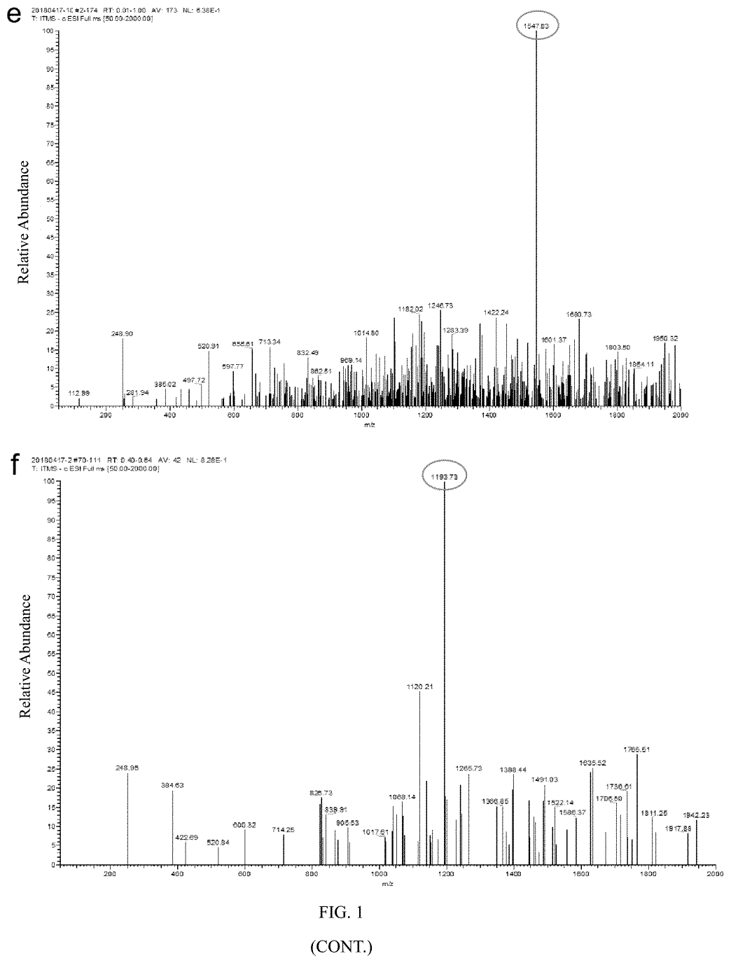

FIG. 4 shows the interaction of CS disaccharide with different proteins in the complement pathway in Example 4.

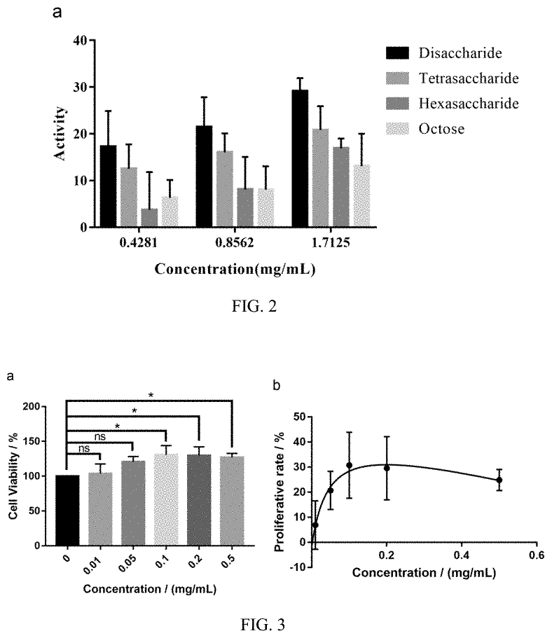

FIG. 5 shows the inhibitory activity of CS disaccharide on the formation of complements C3, C3a, C4b, and C5 in the alternative pathway in Example 5, wherein: a is the inhibitory activity of CS disaccharide on the formation of complement C3 in the alternative pathway, b is the inhibitory activity of CS disaccharide on the formation of complement C3a in the alternative pathway, c is the inhibitory activity of CS disaccharide on the formation of complement C4b in the alternative pathway, and d is the inhibitory activity of CS disaccharide on the formation of complement C5 in the alternative pathway. * P<0.05, ** P<0.01, *** P<0.001, ns: no significant difference.

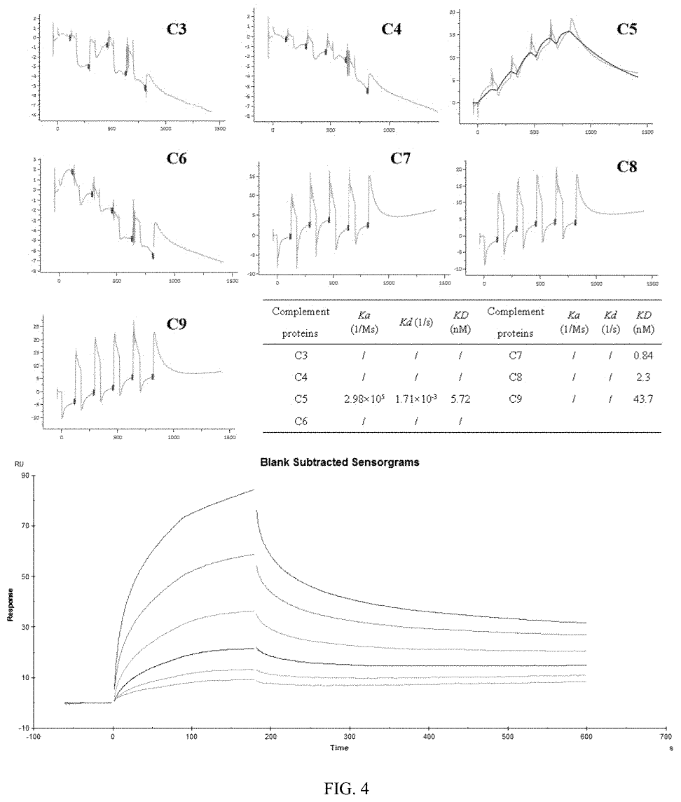

FIG. 6 shows the inhibitory effect of CS disaccharide on MAC formation in the alternative pathway, wherein, a: the effect of different concentrations of CS disaccharide on the level of extracellular lactate dehydrogenase in mouse chondrocytes after NHS stimulation, *** P<0.001, **** P<0.0001; b₁: the MAC level of the blank group, b₂: the MAC level of 10% NHS group, b₃: the MAC level of 10% NHS+0.4 mg/mL CS disaccharide group, b₄: the MAC level of 10% NHS+0.8 mg/mL CS disaccharide group, b₅: the MAC level of 10% NHS+1.2 mg/mL CS disaccharide group.

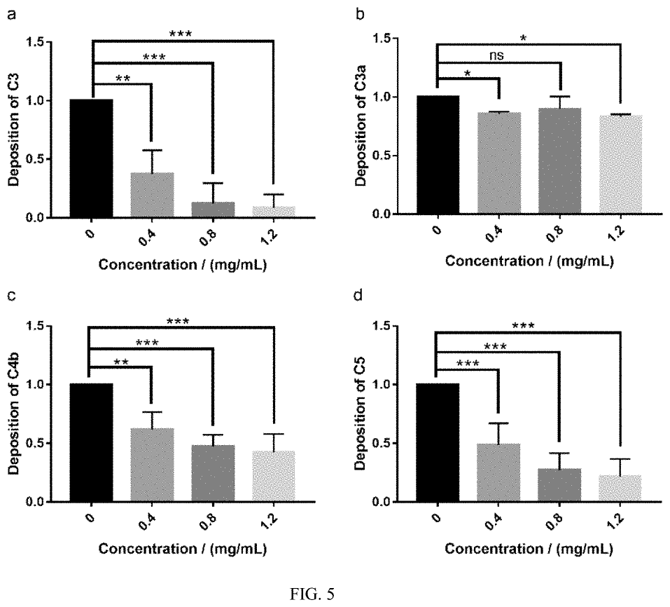

FIG. 7 shows the inhibitory activity of CS disaccharide on MAC-induced expression of related proteins in Example 7, wherein, a: the effect of different concentrations of CS disaccharide on expression of protein MMP-13 in mouse chondrocytes after NHS stimulation, * P<0.05, ** P<0.01; b: the effect of different concentrations of CS disaccharide on expression of protein CCL2 in mouse chondrocytes after NHS stimulation, * P<0.05, ** P<0.01, *** P<0.001; c₁: the imaging results of COX2 in chondrocytes in the blank group, c₂: shows the imaging results of COX2 in chondrocytes in the control group (10% NETS), and c₃: shows the imaging results of chondrocyte in the administered group (10% NHS+0.8 mg/mL CS disaccharide).

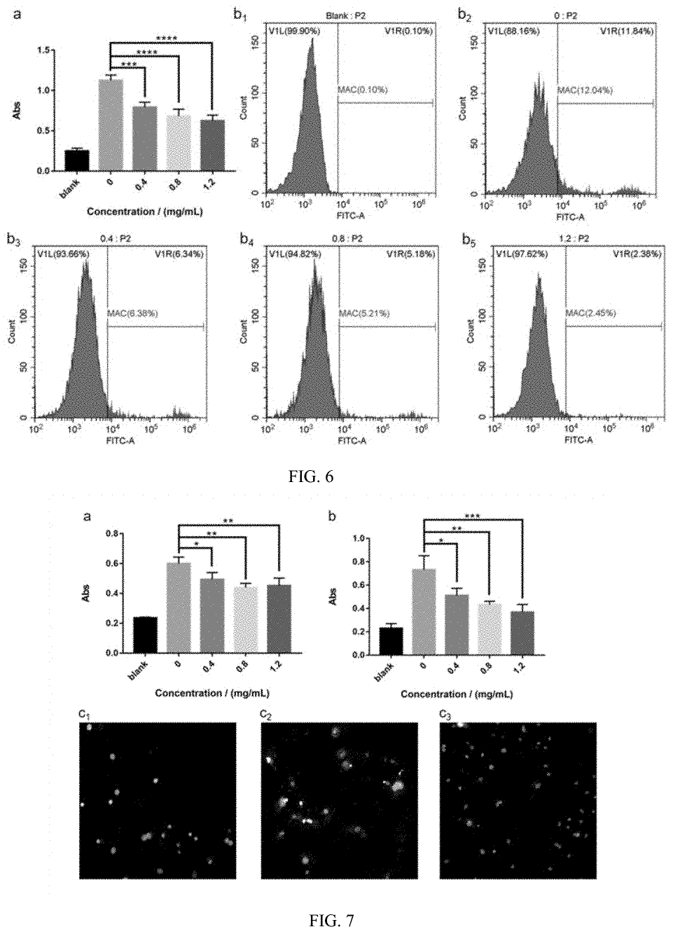

### DETAILED DESCRIPTION

It should be noted that the following detailed descriptions are exemplary and are intended to provide further illustration of the present invention. Unless otherwise indicated, all technical and scientific terms used herein have the same meaning as commonly understood by one of ordinary skill in the art to which the present invention belongs.

It should be noted that the terminology used herein is intended to describe specific embodiments only and is not intended to limit the exemplary embodiments according to the present invention. As used herein, unless the context clearly indicates otherwise, the singular form is also intended to include the plural form, and it is further understood that when the terms “comprising” and/or “including” are used in this specification, they indicate the presence of features, steps, operations, devices, components, and/or combinations thereof. It should be understood that the scope of protection of the present invention is not limited to the specific embodiments described below; it should also be understood that the terms used in embodiments of the present invention are intended to describe specific embodiments and are not intended to limit the scope of protection of the present invention. Experimental methods in the following specific embodiments that do not indicate specific conditions generally follow conventional methods and conditions within the art.

In an exemplary embodiment of the present invention, a method of preparing a CS oligosaccharide with C5 protein targeting comprises:

carrying out enzymatic hydrolysis of CS and separating enzymatic hydrolysis products to obtain different oligosaccharide components;

the oligosaccharide component was verified using hemolysis assay and assay to promote cell proliferation in mice for preliminary investigation and screening of CS oligosaccharides.

In an embodiment of the present invention, the CS is derived from squid.

In another embodiment of the present invention, the enzymatic hydrolysis was performed using CS ABC endolyase (CSase ABC).

In another embodiment of the invention, the enzymatic hydrolysis product is separated by adopting a column chromatography method, for example, such as Bio-Gel P10 column, which was more effective for separating the oligosaccharide components.

In yet another embodiment of the present invention, Bio-Gel P10 column was used to separate the enzymatic hydrolysis products. The mobile phase was 1 M NaCl and 10% ethanol, the flow rate was 2.0 mL/10 min, and the effluent fractions were collected into one tube every 10 min. The detection wavelength was 210 nm, and the same components were combined. The solution was concentrated by centrifugation, desalted with a G25 column, and CS oligosaccharide components (fractions) of different molecular weights were obtained after lyophilization.

In yet another embodiment of the present invention, the method further comprises characterizing the CS oligosaccharides components, the relative molecular mass of the separated CS oligosaccharides component was characterized by electrospray ionization mass spectrometry (ESI-MS).

In yet another embodiment of the present invention, both the hemolysis assay and the assay to promote cell proliferation in mice were performed using conventional experimental methods.

In yet another embodiment of the present invention, the hemolysis assay comprises: taking rabbit erythrocyte suspension and diluting with Me-EGTA buffer; incubating different concentrations of CS oligosaccharide solution with NHS at low temperature with shaking; adding rabbit erythrocyte suspension immediately after the reaction, mixing and incubating.

In yet another embodiment of the present invention, the assay to promote cell proliferation in mice comprises: extracting primary mouse chondrocytes, adding CS oligosaccharide solution with different concentrations, incubating at constant temperature, measuring the number of cells by CCK-8 kit, and calculating the proliferation rate of chondrocytes.

In yet another embodiment of the present invention, the concentration of CS oligosaccharide is 0.4 to 1.2 mg/mL, further preferably 0.4 mg/mL, 0.8 mg/mL or 1.2 mg/mL.

In yet another embodiment of the present invention, the CS oligosaccharides include, but are not limited to, CS disaccharides, CS tetrasaccharides, CS hexasaccharides, CS octasaccharides and CS decasaccharides; preferably CS disaccharide. The CS disaccharide obtained by the preparation and screening method of the present invention was experimentally verified to have optimal C5 protein targeting for the treatment of OA by regulating the complement system.

In yet another embodiment of the present invention, there is provided a CS oligosaccharide, the CS oligosaccharide was obtained by the above preparation method.

The CS oligosaccharides include, but are not limited to, CS disaccharides, CS tetrasaccharides, CS hexasaccharides, CS octasaccharides and CS decasaccharides; preferably CS disaccharide.

In yet another embodiment of the present invention, the application of the above-mentioned CS oligosaccharide in the preparation of OA therapeutic medicines are provided.

The CS oligosaccharides include, but are not limited to, CS disaccharides, CS tetrasaccharides, CS hexasaccharides, CS octasaccharides and CS decasaccharides; preferably CS disaccharide.

The OA therapeutic medicines are particularly medicines for treating OA by regulating complement system.

In order to enable a person skilled in the art to understand the technical solution of the invention more clearly, the technical solution of the invention will be described in detail below in conjunction with specific examples.

### Example 1 Preparation and Characterization of Chondroitin Sulfate (CS) Oligosaccharides

200 μL CS ABC endolyase (CSase ABC) was added to a CS solution of 80 mg/mL. After 18 h, an additional 150 μL of enzyme was added to continue enzymatic hydrolysis for 8 h. After the reaction, the enzyme was inactivated by the method proposed by Sevage and then removed with a 0.22 μm filter. Bio-Gel P10 column (Bio-Rad) was used to separate the enzymatic hydrolysis products. The mobile phase was 1 M NaCl and 10% ethanol, the flow rate was 2.0 mL/10 min, and the effluent fractions were collected into one tube every 10 min. The detection wavelength was 210 nm, and the same components were combined. The solution was concentrated by centrifugation, desalted with a G25 column (Sephadex G-25 Medium, GE Healthcare), and CS oligosaccharide fractions of different molecular weights were obtained after lyophilization. The relative molecular mass of the separated CS oligosaccharides was characterized by electrospray ionization mass spectrometry (ESI-MS) after dissolution with double-distilled water and filtration through a 0.22 μm filter membrane. The MS conditions: mobile phase is 20% water and 80% ethanol, flow rate is 0.2 mL/min, voltage is 3 kV, temperature is 200° C. The results showed that five different oligosaccharide fractions of CS disaccharides, tetrasaccharides, hexasaccharides, octasaccharides and decasaccharides were successfully obtained.

### Example 2 Preliminary Investigation of the Anti-Complement Activity of CS Oligosaccharides by Blood Fusion Method

An appropriate amount of rabbit erythrocyte suspension was taken and washed three times with Mg²⁺-EGTA buffer to make the supernatant colorless and clear, and then the rabbit erythrocytes were diluted to the appropriate concentration with Mg²⁺-EGTA buffer. 80 μL of different concentrations of CS oligosaccharide solution and 10 μL of NHS were added to a V-bottom 96-well plate and incubate on ice for 15 min with shaking. Blank group and negative control group were set up at the same time. Immediately after the reaction, 10 μL of rabbit erythrocyte suspension was added, mixed well, and incubated at 37° C. for 30 min. After the reaction, the supernatant was centrifuged at 2000 r/min for 10 min. 90 μL of the supernatant was collected in a 96-well plate, and the absorbance at 405 nm was measured to calculate the inhibitory activity of CS oligosaccharide on the alternative pathway. The results showed that the anti-complement activity of CS disaccharide was the best compared with other oligosaccharides, while the anti-complement activity of CS octasaccharide was poor. Overall, the anti-complement activity of CS disaccharide was greater than that of CS tetrasaccharide at all concentrations, the anti-complement activity of CS tetrasaccharide was greater than that of CS hexasaccharide, and the anti-complement activity of CS hexasaccharide was greater than that of CS octasaccharide. Moreover, the anti-complement activity of each oligosaccharide basically showed a certain concentration dependence, and the activity gradually increased with the increase of the concentration. After preliminary investigation, the anti-complement activity of CS disaccharide was the best.

### Example 3 CS Promotes Proliferative Activity of Mouse Chondrocytes

The primary C57 mouse chondrocytes were extracted, resuspended in DMEM low glucose medium containing 10% FBS, blown into single cells for counting, and inoculated into 96-well culture plates at a cell density of 3×10³ cells/well in a volume of 100 μL per well, followed by the addition of different concentrations of CS disaccharide solution, and blank group and negative control group were set up at the same time. The 96-well plate was cultured in a constant temperature incubator (5% CO₂, 37° C.) for 24 h. The number of chondrocytes was measured by CCK-8 method, and the proliferation rate of chondrocytes was calculated. The results showed that CS disaccharide could promote the proliferation of mouse chondrocytes to some extent, and in a certain concentration range, the effect of promoting proliferation was enhanced with the increase of concentration.

### Example 4 Interaction of CS Disaccharide with Different Proteins in the Complement Pathway

First, the CS disaccharide was biotinylated. CS disaccharide was dissolved in PBS buffer (pH 7.2) and reacted with Sulfo-NHS-LC-Biotin solution (10 mM) at 4° C. for 2 h. After the reaction, unreacted biotin was removed by the rotate desalination column, and the amount of biotin label was measured by the 4′-hydroxyazobenzene-2-carboxylic acid (HABA) method, Subsequently, the biotinylated CS disaccharide was immobilized on the SA chip. Before the ligand fixation, the chip was pre-treated with 5 μL of 1 M NaCl and 50 mM NaOH for three consecutive times to remove any non-specific contaminants. Biotinylated CS disaccharide (68 nmol/mL, diluted in HBS-EP running buffer at a flow rate of 5 μL/min) and 2 M NaCl were used to wash away unfixed ligand. Finally, 90 of a series of concentrations of complement proteins (C3, C4, C5, C6, C7, C8, C9) were injected into BIAcore 8000 at a flow rate of 30 μL/min, and higher concentrations of complement protein were added every time without regeneration. The kinetic relationship between complement-related protein and CS disaccharide was investigated. The results showed that CS disaccharide bound to various complement proteins to some extent, wherein C5 showed the strongest interaction with CS disaccharide; the KD calculated was 5.72×10⁻⁹ M, which indicates very tight binding between the molecules, further verifying that CS disaccharide has strong C5 protein targeting.

### Example 5 CS Disaccharide Inhibits the Formation of C3, C3a, C4b, and C5 Complements of the Alternative Pathway

50 μL CS disaccharide solution was prepared with HMEBN solution containing 20% NHS at different concentrations, and three concentration gradients (400 μg/mL, 800 μg/mL, 1200 μg/mL) were set up, and control (without CS disaccharide) and blank (without CS disaccharide and NETS) groups were incubated at 37° C. for 1 h. 40 μL of sample dilution was added to the enzyme-coated plate, followed by 10 μL of the sample to be tested (final dilution of the sample was 5 times). 40 μL of sample dilution was added to the enzyme-coated plate, followed by 10 μL of the sample to be tested (final dilution of the sample was 5 times) was added, and the plate was sealed with a sealing film and incubated at 37 for 30 min. The sealing film was carefully removed, the liquid was discarded, shaken dry, each well was filled with washing buffer, left for 30 s and discarded, repeat the above operation 5 times and tapped dry. 50 μL of enzyme standard reagent was added to each well, except for the blank wells. The plate was sealed with a sealing film and incubated at 37° C. for 30 min, then washed. 50 μL of reagent A was first added to each well, and then 50 μL of reagent B was added, shaken gently, and mixed evenly. The reaction was developed at 37° C. without light for 10 min, and 50 μL of termination solution was added to each well to stop the reaction (the blue color turned yellow at this time). 15 min later, the absorbance of each well was measured sequentially at 450 nm wavelength with blank wells adjusted to zero. The results showed that different concentrations of CS disaccharide could inhibit the formation of C3, C3a, C4b and C5 to some extent. From the overall, almost all dose groups showed significant inhibition compared with the control group, and the inhibitory activity was positively correlated with the concentration of CS disaccharide, further indicating that CS disaccharide has good inhibitory activity on complement formation of alternative pathway and strong targeting of C5 protein.

### Example 6 CS Disaccharide Inhibition of MAC Formation in the Complement Pathway

The level of lactate dehydrogenase in the supernatant was measured by ELISA, and the experimental procedure is described in Example 5.

The chondrocytes were seeded into a 6-well culture plate at a cell density of 1.5×10⁶ cells per well, added with different concentrations of CS disaccharide, and cultured for 48 h. After digestion with trypsin, the cells were scraped off and transferred to 1.5 mL ep tube in dark. Cells were washed twice with serum-free PBS to remove trypsin, and 100 μL of PBS containing 10% FBS was added to each tube to resuspend the cells. 50 μL of primary antibody (Anti-05b-9 antibody (aE11)) was added to each tube so that the concentration of primary antibody ranged from 0.1 μg/mL to 10 μg/mL and incubated at 4° C. in the dark, followed by washed with three times, centrifuged at 400 g/min for 5 min, resuspended in ice-cold PBS, 50 μL of secondary antibody (FITC-labeled goat anti-mouse IgG) was added to each well, incubated at 4° C. in the dark for 20-30 min, washed three times, centrifuged at 400 g/min for 5 min, resuspended in ice-cold PBS and immediately transferred to a dark environment at 4° C. The analysis was performed using a flow cytometer (CytoFLEX S, Beckman Coulter, Inc.). Anti-05b-9 antibody (aE11) was purchased from Abcam, and FITC-labeled goat anti-mouse IgG was purchased from Shanghai Yisheng Biotechnology Co., Ltd.

The above two experiments demonstrate that NHS induced the formation of the MAC, which can be inhibited by CS disaccharide, and the inhibitory effect was positively correlated with the concentration of the CS disaccharide used.

### Example 7 CS Disaccharide Inhibits the Expression of MAC-Induced Proteins

The study of CCL2 and MMP13 in the example was performed by ELISA, and the experimental operation was described in Example 5.

The study of COX2 in the example was performed by immunocytochemical assay. The chondrocytes were inoculated into a 6-well culture plate at a density of 1×10⁶ cells per well, cultured for 24 h and administered after cell adhesion. The medium was discarded and the cells were subsequently covered with a layer of 2-3 mm of 4% formaldehyde diluted with PBS and fixed at room temperature for 15 min. The fix solution was discarded and the cells were rinsed three times with PBS. The specimens were blocked in a blocking buffer for 60 min. The blocking buffer was discarded and diluted primary antibody (COX2 (D5H5) XP rabbit monoclonal antibody) was added, and incubated overnight at 4° C., rinsed three times with PBS, secondary antibody (biotinylated goat anti-rabbit IgG (H+L)) labeled with fluorescent substance diluted in antibody dilution buffer was added and incubated for 1-2 h at room temperature in the dark, washed three times with PBS, a small amount of DAPI staining solution was added (just cover the sample), stained for 10 min at room temperature, and washed 3 times with PBS buffer. The stained cells were photographed by laser confocal high content imaging analysis system (PerkinElmer). Cox2 (D5H5) XP rabbit monoclonal antibody was purchased from Cell Signaling technology, and biotinylated goat anti-rabbit IgG (H+L) was purchased from Shanghai Yisheng Biotechnology Co., Ltd.

The results showed that CS disaccharide inhibited the complement pathway and thereby downregulated the MAC-induced expression of MMP13, CCL2 and COX2.

It should be noted that the above examples are used only to illustrate the technical solution of the present invention and not to limit it. Although the invention is described in detail with reference to the examples given, a person of ordinary skill in the art may modify or make equivalent substitutions to the technical solutions of the invention as needed without departing from the spirit and scope of the technical solutions of the invention.

1. A method for preparing chondroitin sulfate oligosaccharides with C5 protein targeting, wherein the method comprises:
carrying out enzymatic hydrolysis of chondroitin sulfate and separating enzymatic hydrolysis products to obtain different oligosaccharide components;
verifying the oligosaccharide component using hemolysis assay and cell proliferation assay for preliminary investigation and screening of chondroitin sulfate oligosaccharides.

2. The method for preparing chondroitin sulfate oligosaccharides with C5 protein targeting as claimed in claim 1, wherein the chondroitin sulfate is derived from squid.

3. The method for preparing chondroitin sulfate oligosaccharides with C5 protein targeting as claimed in claim 1, wherein the enzymatic hydrolysis is performed using chondroitin sulfate ABC endolyase.

4. The method for preparing chondroitin sulfate oligosaccharides with C5 protein targeting as claimed in claim 1, wherein the enzymatic hydrolysis product is separated by adopting a column chromatography method, the column used is in column chromatography is Bio-Gel P10 column;
separating the enzymatic hydrolysis products comprises: using 1 M NaCl and 10% ethanol as mobile phase, and separating the enzymatic hydrolysis products by using Bio-Gel P10 column with a flow rate of 2.0 mL/10 min, collecting the effluent fractions into one tube every 10 min, combining the same components with a detection wavelength of 210 nm; concentrating by centrifugation, desalting with a G25 column, and lyophilizing to obtain chondroitin sulfate oligosaccharide components of different molecular weights.

5. The method for preparing chondroitin sulfate oligosaccharides with C5 protein targeting as claimed in claim 1, wherein the method further comprises characterizing the oligosaccharide components, the relative molecular mass of the oligosaccharide component is characterized by electrospray ionization mass spectrometry.

6. The method for preparing chondroitin sulfate oligosaccharides with C5 protein targeting as claimed in claim 1, wherein the hemolysis assay comprises: taking rabbit erythrocyte suspension and diluting with Mg²⁺-EGTA buffer; incubating different concentrations of chondroitin sulfate oligosaccharide solution with NHS at low temperature with shaking; adding rabbit erythrocyte suspension immediately after the reaction, mixing and incubating.

7. The method for preparing chondroitin sulfate oligosaccharides with C5 protein targeting as claimed in claim 1, wherein the cell proliferation assay comprises:
extracting primary mouse chondrocytes, adding chondroitin sulfate oligosaccharide solution with different concentrations, incubating at constant temperature, measuring the number of cells by CCK-8 kit, and calculating the proliferation rate of chondrocytes.

8. The method for preparing chondroitin sulfate oligosaccharides with C5 protein targeting as claimed in claim 1, wherein the chondroitin sulfate oligosaccharides comprise chondroitin sulfate disaccharides, chondroitin sulfate tetrasaccharides, chondroitin sulfate hexasaccharides, chondroitin sulfate octasaccharides and chondroitin sulfate decasaccharides.

9. A chondroitin sulfate oligosaccharide prepared by the method as claimed in claim 1.

10. Application of the chondroitin sulfate oligosaccharide as claimed in claim 9 in the preparation of osteoarthritis therapeutic medicines;
the chondroitin sulfate oligosaccharides comprise chondroitin sulfate disaccharides, chondroitin sulfate tetrasaccharides, chondroitin sulfate hexasaccharides, chondroitin sulfate octasaccharides and chondroitin sulfate decasaccharides;
the osteoarthritis therapeutic medicines are medicines for treating osteoarthritis by regulating complement system.
